(19) United States
(12) Patent Application Publication
(10) Pub. No.: US 2016/0263387 A1
(43) Pub. Date: Sep. 15, 2016

(54) RADIOFREQUENCY TREATMENT PROBE FOR TREATING VAGINAL LAXITY AND ASSOCIATED SYSTEMS AND METHODS

(71) Applicant: ThermiGen, LLC, Southlake, TX (US)

(72) Inventors: Red Alinsod, Laguna Beach, CA (US); Paul R. Herchman, JR., Grapvevine, TX (US); Kevin D. O'Brien, Coppell, TX (US)

(21) Appl. No.: 14/641,435

(22) Filed: Mar. 9, 2015

(51) Int. Cl. A61N 1/40; A61N 1/08

(52) U.S. Cl. CPC A61N 1/403; A61N 1/08

(57) ABSTRACT

The present invention is related to a radiofrequency treatment probe and method delivering radiofrequency for controlled heating of tissue within the vagina for the treatment of vaginal laxity. The treatment probe may include a curved, rounded disposable treatment tip that may be designed to accommodate vaginal anatomy. An electrode assembly may be coupled to the treatment tip, wherein the electrode coupled to the treatment tip may be configured to transfer radiofrequency energy through vaginal skin surface to the tissue. A temperature measuring feature may include or be coupled to the electrode assembly, wherein the electrode assembly may be configured to regulate and maintain a pre-determined skin temperature. A radiofrequency handle may be configured to connect to a disposable treatment tip. A connector may be configured to connect to the radiofrequency handle to a radiofrequency generator. A protective apparatus may be configured to protect the radiofrequency handle.

### RELATED APPLICATIONS

This application is a continuation-in-part and claims the benefit under 35 U.S.C. §120 of U.S. patent application Ser. No. 14/591,002 filed on Jan. 7, 2015, which claimed the benefit of U.S. provisional patent 61/982,590 filed on Apr. 22, 2014.

### FIELD OF THE INVENTION

The present invention relates to systems and methods for treating vaginal laxity and, more specifically, to treating vaginal laxity by inducing collagen growth.

### BACKGROUND

Vaginal laxity, also referred to as pelvic organ prolapse (POP), is defined as a condition in which vaginal wall support is lost, and in more severe cases, various pelvic organs prolapse into the vagina. This can have a serious impact on a woman's quality of life, self-esteem, and sexual enjoyment. Furthermore, vaginal laxity may contribute to urinary stress incontinence.

Vaginal laxity can be caused by a change within the collagen composition, thereby causing a weaker pelvic floor. Additionally, vaginal laxity may result from the traumatic expansion of the vaginal wall during vaginal birth.

A histological sample of the vaginal wall is depicted in FIG. 4. The vagina is lined by Stratified Squamos Epithelium 90 that features a small degree of keratinization on its Surface 95. Below the epithelium is a thick layer of dense Connective Submucosa Tissue 93, like that in the dermis of the skin. A layer of loose connective tissue containing many blood vessels and nerves lies further underneath this.

Connective tissue aberrations or deformities have been identified as a predominant cause of vaginal laxity, as the connective tissues provide critical pelvic floor support. Briefly, connective tissue is composed of collagen, elastin, smooth muscles, and microfibers. Studies have reported connective tissue defects predispose women to POP, and in turn, vaginal laxity. Women diagnosed with POP have demonstrated less total collagen content in the supportive pubocervical fascia, along with a weaker collagen matrix. Evaluation of vaginal tissue from women with and without prolapse has shown changes in expression of collagen type and elastin metabolism, an indication that vaginal tissue remodels in response to POP-induced mechanical stresses. Additionally, age and neuroendocrine changes disintegrate connective tissue strength, which in turn, predisposes patients to vaginal laxity.

At present, there are two ways of dealing with POP. The first method for treatment involves surgery, which can be vaginal or abdominal. Surgical intervention is typically undesirable due to cost, pain and suffering to the patient and the possibility that even surgery will fail to be effective. Efforts to avoid surgical procedures have resulted in the development of a number of non-surgical vaginal devices, inserted into the vagina by the surgeon or the patient. Therefore, the second method of treatment requires the use of vaginal devices (pessaries) that are inserted into the vagina and mechanically reduce the prolapse by pushing the vaginal walls aside and upwards. Vaginal devices are well known for their tremendous diversity in shapes and sizes.

Some of these devices tend to diminish urine flow from the bladder. Therefore, when a patient needs to urinate, the device must be removed from the vagina or must be collapsed to remove the pressure applied against the bladder neck. In an attempt to solve this problem, vaginal devices were developed in special shapes that do not completely block the bladder neck so that the patient may urinate with the device in place. These devices, however, are generally large and intrusive and, therefore, are uncomfortable to insert, wear, and remove, resulting in low patient satisfaction and compliance. They are also relatively expensive, and therefore designed to be reusable. Various pessary devices have been designed to treat prolapse in women, for example, U.S. Pat. No. 6,189,535; U.S. Pat. No. 6,158,435; U.S. Pat. No. 5,894,842; U.S. Pat. No. 5,771,899; U.S. Pat. No. 5,611,768; U.S. Pat. No. 4,823,814; and GB 19124034, the disclosures of which are herein incorporated by reference.

To overcome limitations of the aforementioned devices and methods, the industry needs a method to promote connective tissue remodeling, and reinforce collagen strength to avoid the use of pessaries by reinforcing vaginal anatomy and correcting vaginal laxity.

This background information is provided to reveal information believed by the applicant to be of possible relevance to the present invention. No admission is necessarily intended, nor should be construed, that any of the preceding information constitutes prior art against the present invention.

### SUMMARY OF THE INVENTION

With the above in mind, embodiments of the present invention are related to a radiofrequency treatment probe and method for treating a number of conditions, including but not limited to:

a. vaginal laxity;
b. pelvic organ prolapse;
c. incontinence;
d. tissue tone, including moisture, and tightness;
e. nerve sensitivity and orgasmic dysfunction;
f. vulvovaginal laxity improvement in tightening the labial and vaginal tissues;
g. reduction in atrophic vaginitis;
h. reduction in stress incontinence and pelvic organ prolapse by tightening the pubocervical fascia; and,
i. treatment for orgasmic dysfunction by improving nerve sensitivity of vulvovaginal structures including clitoral nerves and branches.

One embodiment of the treatment probe may include a curved, rounded treatment tip that may be designed to accommodate vaginal anatomy. An electrode assembly may be coupled to the treatment tip, wherein the electrode coupled to the treatment tip may be configured to transfer radiofrequency energy to specific vaginal structures. A temperature measuring feature may be coupled to the electrode assembly, wherein the temperature measuring feature that may be coupled to the electrode assembly may be configured to monitor and regulate electrode and skin temperature. A radiofrequency handle may be configured to connect to the treatment tip. A connector may be configured to connect the radiofrequency handle to a radiofrequency generator. A protective apparatus may be configured to protect the radiofrequency handle.

In another embodiment, the treatment probe may include a curved, rounded disposable treatment tip that may be designed to accommodate vaginal anatomy. An electrode assembly may be coupled to the treatment tip, wherein the electrode coupled to the treatment tip may be configured to transfer radiofrequency energy to specific vaginal structures. A temperature measuring feature may be coupled to the electrode assembly, wherein the temperature measuring feature coupled to the electrode assembly may be configured to monitor and regulate electrode and skin temperature. A reusable radiofrequency handle may be configured to connect to the treatment tip. A connector may be configured to connect the radiofrequency handle to a radiofrequency generator. A disposable protective apparatus may be configured to protect the radiofrequency handle. The treatment tip may be excited by a frequency, a power level, and pattern configured to operate within a vulvovaginal cavity for the purpose of treating vulvovaginal laxity. The temperature-measuring feature may be configured to use a thermocouple or other similar device.

In yet another embodiment of the invention, a method of treating vaginal laxity with a treatment probe may include the steps of cleansing the specific vaginal structure, applying the treatment tip along the specific vaginal structure, repeating the applying the treatment tip along the specific vaginal structure, and generating power with the radiofrequency generator.

Throughout this application, the treatment tip has been assumed to be a small specific area at the end of the treatment probe, but the treatment area is limited only by the number of RF-excitable areas. In one embodiment the entirely of the treatment probe could be covered in RF-excitable areas, such that a user need not move the probe in order to treat a particular area, but can merely excite that area of interest for the desired effect on the patient.

The bulk of this disclosure concerns the use of RF energy to heat tissue, but this is but one means of heating tissue and the invention encompasses other means, including localized microwave, sonic, and other approaches.

### BRIEF DESCRIPTION OF THE DRAWINGS

FIG. 1 is an exploded view of a device according to an embodiment of the present invention.

FIG. 2 shows the control scheme of the invention as currently embodied.

FIG. 3A and FIG. 3B are top and side views of a current embodiment of the Interior Portion 1 from FIG. 1.

FIG. 4 shows a section of vaginal wall for explanatory purposes.

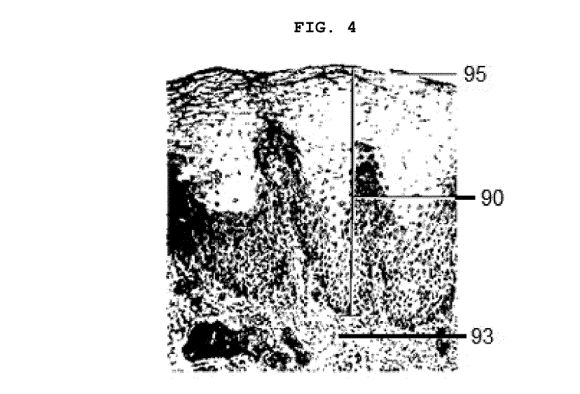

FIG. 5 is a diagram of a distribution of radiofrequency induced heat according to an embodiment of the present invention.

FIG. 6 is a diagram of the front panel of a radiofrequency generator according to an embodiment of the present invention.

FIG. 7 is a diagram of a system for treating vaginal laxity according to an embodiment of the present invention.

FIG. 8 is a schematic of a radiofrequency generator and associated components according to an embodiment of the present invention.

FIGS. 9A and 9B are alternative embodiments of the invention.

### DETAILED DESCRIPTION OF THE INVENTION

The present invention will now be described more fully hereinafter with reference to the accompanying drawings, in which preferred embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art. Those of ordinary skill in the art realize that the following descriptions of the embodiments of the present invention are illustrative and are not intended to be limiting in any way. Other embodiments of the present invention will readily suggest themselves to such skilled persons having the benefit of this disclosure. Like numbers refer to like elements throughout.

Although the following detailed description contains many specifics for the purposes of illustration, anyone of ordinary skill in the art will appreciate that many variations and alterations to the following details are within the scope of the invention. Accordingly, the following embodiments of the invention are set forth without any loss of generality to, and without imposing limitations upon, the claimed invention.

In this detailed description of the present invention, a person skilled in the art should note that directional terms, such as “above,” “below,” “upper,” “lower,” and other like terms are used for the convenience of the reader in reference to the drawings. Also, a person skilled in the art should notice this description may contain other terminology to convey position, orientation, and direction without departing from the principles of the present invention.

Furthermore, in this detailed description, a person skilled in the art should note that quantitative qualifying terms such as “generally,” “substantially,” “mostly,” and other terms are used, in general, to mean that the referred to object, characteristic, or quality constitutes a majority of the subject of the reference. The meaning of any of these terms is dependent upon the context within which it is used, and the meaning may be expressly modified.

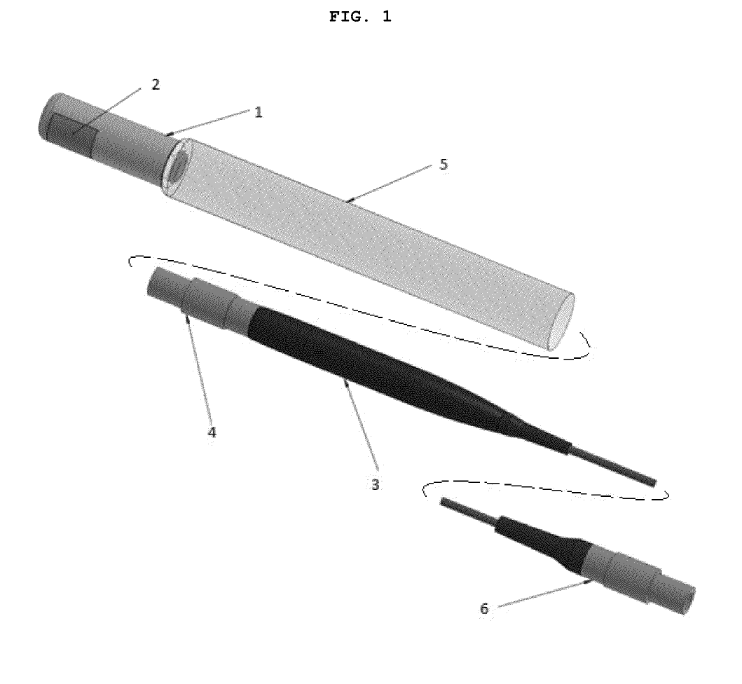

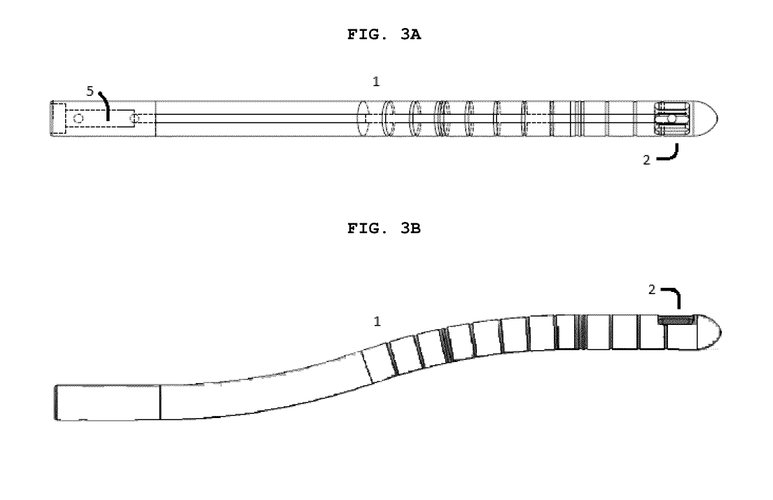

Referring now to FIG. 1, a device, according to an embodiment of the present invention, is now described in detail. FIG. 1 represents a previous embodiment of the invention. FIG. 3A and FIG. 3B represent the current and preferred embodiment of the present invention and have replaced FIG. 1. However, detailed discussion of FIG. 1 remains beneficial. The device may comprise an Interior Portion 1 (potentially disposable), a Radiofrequency (hereinafter referred to as “RF”) Electrode Assembly 3, and an RF Source 6. The Interior Portion 1 may comprise an active Treatment Electrode 2 and a Connecter 5. The RF Electrode Assembly 3 may be coupled to the Interior Portion 1 by the Connector 5. The RF Electrode Assembly 3 may further be coupled to an RF generator by the RF Source 6. In one embodiment of the invention, the Connector 5 may be coupled to an RF Connection Handle 4 (potentially disposable).

In one embodiment, the RF Electrode Assembly (interchangeably referred to as the Electrode Assembly) may comprise a conductive portion, a dielectric portion, a radiofrequency electrode, and a flex circuit. The radiofrequency electrode may be configured to capacitively couple radiofrequency energy with vaginal tissue when at least one of the conductive portion and the dielectric portion is in contact with a skin surface. The flex circuit may be coupled to the radiofrequency electrode and may be configured so that the conductive portion comprises a plurality of voids. The dielectric portion may be configured to be positioned between the conductive portion and the skin surface when the radiofrequency electrode is positioned at the skin surface.

The electrode assembly may further comprise a back plate and a plurality of electrical contact pads. The back plate may be coupled to a support structure and the plurality of electrical pads may be coupled to the back plate. The support structure may comprise a first engagement member and a second engagement member. The first and second engagement members may be configured to provide engagement and disengagement with a hand-piece support structure.

The Interior Portion 1 may also comprise a temperature measuring feature and a protective apparatus. In one embodiment of the invention, the protective apparatus may encase the Interior Portion 1. The protective apparatus may comprise at least one of flexible latex and solid acrylic material.

In another embodiment of the invention, the temperature measuring feature may be configured to be positioned along a back surface of a radiofrequency electrode of the Interior Portion 1. The temperature measuring feature may be configured to detect temperature along the back side of the radiofrequency electrode.

In one embodiment of the invention, the temperature measuring feature of Interior Portion 1 may comprise a non-invasive, transmucosal treatment probe. The transmucosal treatment probe may be configured to elevate mucosal tissue temperatures to about 40-45° C. for the purpose of promoting tissue contracture. Additionally, real-time temperature monitoring may be carried out using a thermocouple. In one embodiment, the thermocouple may be integrated with a thermistor treatment probe. The thermistor may be configured to use a feedback mechanism. The feedback mechanism may be configured to integrate a central processing unit to turn off the radiofrequency electrode of the Interior Portion 1 when temperatures exceed a preset temperature. In the current embodiment, the efficacy of the thermocouple or other temperature-measuring device may be configured to be most effective with manual movement of the Interior Portion 1 not exceeding one centimeter per second, but other embodiments may require different speeds.

In another embodiment of the invention, the device may be vaginally inserted. The treatment tip may be applied to labial tissues then perineum then introitus then up to hymeneal ring the entire depth of the vagina to the apex. The application may start at the perineal body, then the introitus, and then up to hymeneal ring and then deeper, treating the entire vaginal mucosal surface.

In yet another embodiment of the invention, the treatment tip may be applied to a full length of a vaginal canal. In this embodiment, the treatment tip may be applied to the labia majora and minora tissue, lateral clitoral hood, perineum, introitus, vaginal wall to apex of the vagina, and other regions of the full length of the vaginal canal concurrently.

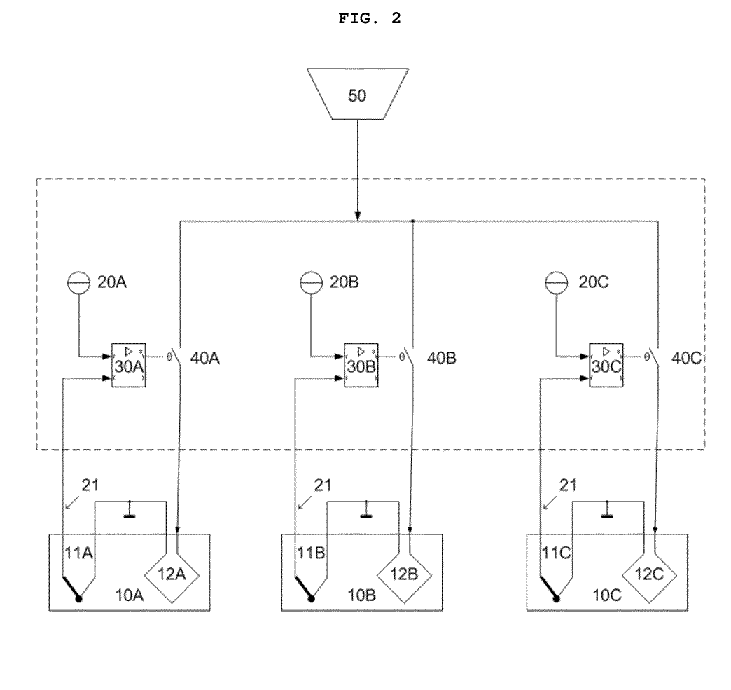

Referring now to FIG. 2, a control scheme, according to an embodiment of the present invention, is now described in detail. The control scheme may maintain an electrode set temperature during a treatment phase.

As an illustration of how a circuit functions, the disclosed embodiment may comprise a first Electrode 10A, a second Electrode 10B, and a third Electrode 10C. It should be understood that this embodiment permits temperature control of a plurality of electrodes. In other embodiments, there may be two, three, four, or more different electrodes controlled by the control scheme, so that the several electrodes that be modulated to impact an individual area temperature without having to move the device, as the entire handpiece is one array of electrodes.

As seen in FIG. 2, each Electrode 10A 10B 10C (hereinafter referred to as “10”) may include an incorporated Temperature Sensor 11A 11B 11C (hereinafter referred to as “11”). In one embodiment of the present invention, the Temperature Sensor 11 may report the temperature at an electrode tip. Furthermore, the Temperature Sensor 11 may apply a high frequency energy to the Electrodes 10. In one embodiment, the high frequency energy may be applied using an industry available RF Tip 12A 12B 12C (hereinafter referred to as “12”). Temperature may be reported through Sensor Lines 21 from each Electrode 10 to each Control Unit 30A 30B 30C (hereinafter referred to as “30”). Each Control Unit 30 may also comprise an Input Set Temperature 20A 20B 20C (hereinafter referred to as “20”). In one embodiment, the Control Unit 30 may compare the Input Set Temperature 20 to a temperature reported by the Temperature Sensor 11. In this embodiment, the Control Unit 30 may be configured to further determine whether to open or close a Switch 40A 40B 40C (hereinafter referred to as “40”). In the current embodiment, the Switch 40 is electrical. However, those of ordinary skill in the art understand that the Switch 40 is not limited to an electrical switch. The Switch 40 may comprise a mechanical, optical, or any number of other control mechanism known in the industry.

A RF Energy Input 50 may be connected and disconnected to each Electrode 10 through the Switch 40. The connection through the Switch 40 may be operated by the Control Unit 30. In the present embodiment, the operation of the Switch 40 by the Control Unit 30 may be achieved by comparing the Input Set Temperature 20 to a temperature reported at the Temperature Sensor 11. The Control Unit 30 may be configured to deliver additional RF energy from the RF Energy Input 50 as a result of the comparison between the Input Set Temperature 20 to the temperature reported by the Temperature Sensor 11. In one embodiment, the Control Unit 30 may be configured to deliver additional RF energy so that the temperature at the Temperature Sensor 11 is approximately the Input Set Temperature 20. One of ordinary skill in the art will recognize that there are various algorithms for feedback circuits and each may be used by the Control Unit 30 to accomplish substantially the same result described in the embodiment above. One of ordinary skill in the art will also recognize that a plurality of methods exist to provide energy through heat. In another embodiment of the present invention, the RF energy may be replaced with ultrasound or other types of excitation.

Referring now to FIG. 3A and FIG. 3B, a disposable interior portion of the device discussed in FIG. 1, according to an embodiment of the present invention, is described in detail. The disposable Interior Portion 1 of FIGS. 3A and 3B is the Interior Portion 1 of the device of FIG. 1. The Interior Portion 1 comprises an active Treatment Electrode 2 and a Connector 5. The Interior Portion 1 may further comprise a treatment tip. In one embodiment of the present invention, the active Treatment Electrode 2 may be a ThermiVa Electrode. In one embodiment of the present invention, the treatment tip may be a ThermiVa Tip. In yet another embodiment, RF energy may be delivered to the Interior Portion 1 via handheld treatment probe.

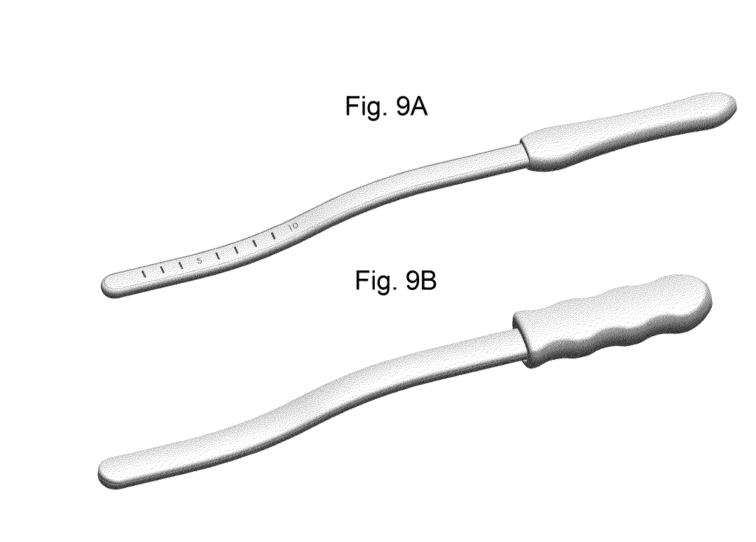

FIGS. 9A and 9B show alternative embodiments of the invention, particularly showing the Interior Portion, showing the shape of the device.

In one preferred embodiment of the present invention, the Interior Portion 1 may employ a slight S-curve shape to follow natural vaginal curves and provide a comfortable shape to a user. Additionally, in one embodiment of the present invention, the handheld treatment probe and the Interior Portion 1 may comprise a larger treatment surface. The larger surface may extend up to the entire device generating at least one of RF heat and ultrasound heat into all surfaces of the vaginal canal.

In one embodiment of the present invention, treatment of all surfaces of the vaginal canal, including, but not limited to, the labia majora and labia minora, may result in reduction of labial laxity. The RF heat, as well as the other heat sources, may result in tissue coagulation and tightening and encouragement of new collagen to form. Additionally, in another embodiment of the present invention, treatment of atrophic vaginitis may be possible due to collagen stimulation.

In another embodiment of the present invention, collagen effects on healing may result in reducing pelvic pain and vaginismus. In yet another embodiment of the present invention, at least one of tightening of periurethral tissues, improved coordination of muscle contractions, and stronger muscle contractions of both periurethral muscles and urethral muscles may result in reduction of stress incontinence.

In another embodiment of the present invention, at least one of tightening of posterior, anterior and sidewalls of the vaginal canal and pubocervical and rectovaginal fascias may result in reduction of pelvic prolapse symptoms. In yet another embodiment of the present invention, at least one of tightening of anal mucosa and stimulation of sphincter muscles may result in reduction of anal incontinence.

In another embodiment of the present invention, the RF heat, as well as other types of energy through heat, may result in decreased orgasmic dysfunction and improvement in nerve sensitivity.”. One of ordinary skill in the art can recognize that above are just some potential examples of various embodiments of the use of heat through energy. One of ordinary skill in the art can also recognize that many more treatments may benefit from the application of energy through heat as applied by the device discussed hereinabove.

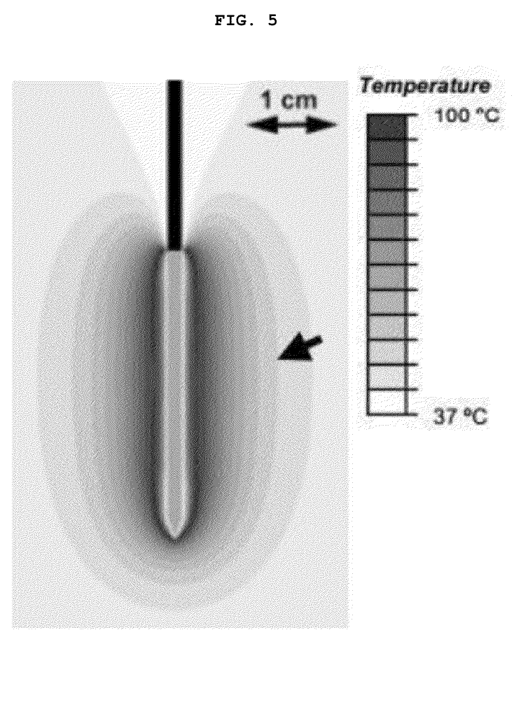

Referring to FIG. 5, a distribution of RF-induced heat, according to an embodiment of the present invention, is now described in detail. Transcutaneous RF may provide a treatment for tissue contracture. Heat from a transcutaneous temperature controlled radiofrequency device may promote neo-collagenesis, denaturation of collagen cross-links, activation of wound healing pathways, contraction of collagen, and increasing in collagen fibril size.

The heat may be the result of RF experience impedance as current traverses a tissue bed. As electric current permeates a tissue layer, ions found within that tissue layer may deliver the electric current. As a result, there may an increase in kinetic activity of the ions. Increased ion kinetics and oscillations may engender resistive tissue thermogenesis. Thermogenesis may be calculable via the Specific Absorption Rate (hereinafter referred to as “SAR”) equation. SAR assesses local electrical conductivity and magnitude of local electric current density generated around an electrode.

A therapeutic benefit of the heat may be localized thermogenesis. An electric field strength generated by the RF energy may be capable of heating tissue in close proximity to the electrode. Thermal conduction may attenuate the heating of the tissue as you move further from the electrode. With proper power controls, a generated ideal thermal endpoint may occur close to the electrode. As a result, only the desired specific tissue may be affected.

Regulation of tissue temperature may derive from power control. Power in an embodiment of the present invention may be electrical voltage delivered to an RF electrode. Depending on specific tissue impedance, the power may need to be adjusted to ensure that a proper voltage is delivered to satisfy the specific tissue impedance.

Thermal sensors, for example, but not necessarily limited to, may comprise at least one of thermocouples, thermistors, or other temperature-measuring device. The thermal sensors may be integrated within the RF electrode to adjust power to maintain a desired therapeutic temperature. In one embodiment, a thermal camera may provide real-time skin temperature monitoring. With proper controls in place, selective thermogenesis may serve as a viable treatment for numerous medical conditions.

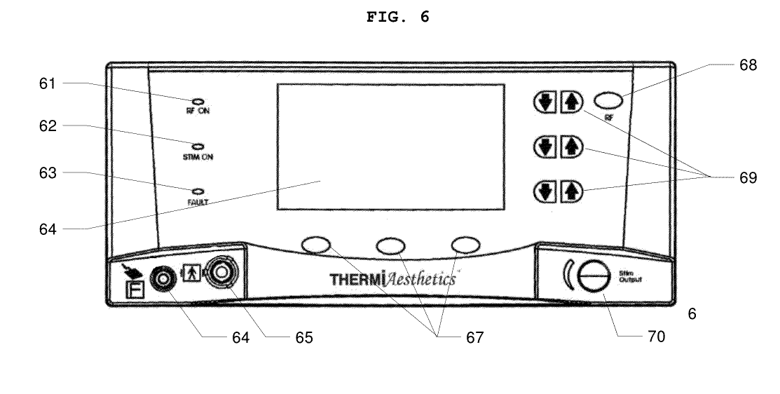

Referring to FIG. 6, an RF Source 6 or RF generator, according to an embodiment of the present invention, is now described in detail. This document will use ‘RF Generator’ and ‘RF Source’ interchangeably, as the RF generator of FIG. 6 is one embodiment of the RF Source of FIG. 1.

The RF Source 6 may comprise an RF On Light 61, a STIM On Light 62, a Fault Light 63, a Neutral Electrode Connection Port 64, a Device Connection Port 65, a Display Window 66, a plurality of Soft Keys 67, an RF On Button 68, a plurality of Up/Down Buttons 69, and a Stim Output Knob 70.

The RF On Light 61 may illuminate when the RF Generator 6 is delivering RF power. The Stim On Light 62 may illuminate when the RG generator is delivering stimulate power. The Fault Light 63 may illuminate when a fault condition is detected. The Neutral Electrode Connection Port 64 may be used to connect a neutral electrode to the RF generator. The Device Connection Port 65 may be used to connect devices to the RF Generator 6. The devices may comprise ThermiAesthetics devices among other devices.

The Display Window 66 may display a plurality of information that may comprise, but is not limited to, RF generator information, modes of operation, and operating parameters. The plurality of Soft Keys 67 may comprise, but are not limited to, STIM: Motor, ThermiTight, ThermiSmooth, ThermiRase, Help, Exit, Start, Reset, Ok, and other keys. The RF On Button 68 may, upon being pressed, start or stop RF power delivery. The plurality of UP/Down Buttons 69 may be used, upon being pressed, to increase function settings, among other operations. The Stim Output Knob 70 may adjust a stimulate output voltage. Additionally, the Stim Output Knob 70, when pressed and released, may turn stimulate power on and off.

In one embodiment of the present invention, the RF Source 6 referenced in FIG. 1 and shown in FIG. 6 may comprise the ThermiRF RF generator. Those skilled in the art will readily appreciate, however, that other RF generators may be configured in a way to still accomplish the many goals, features and advantages according to the present invention.

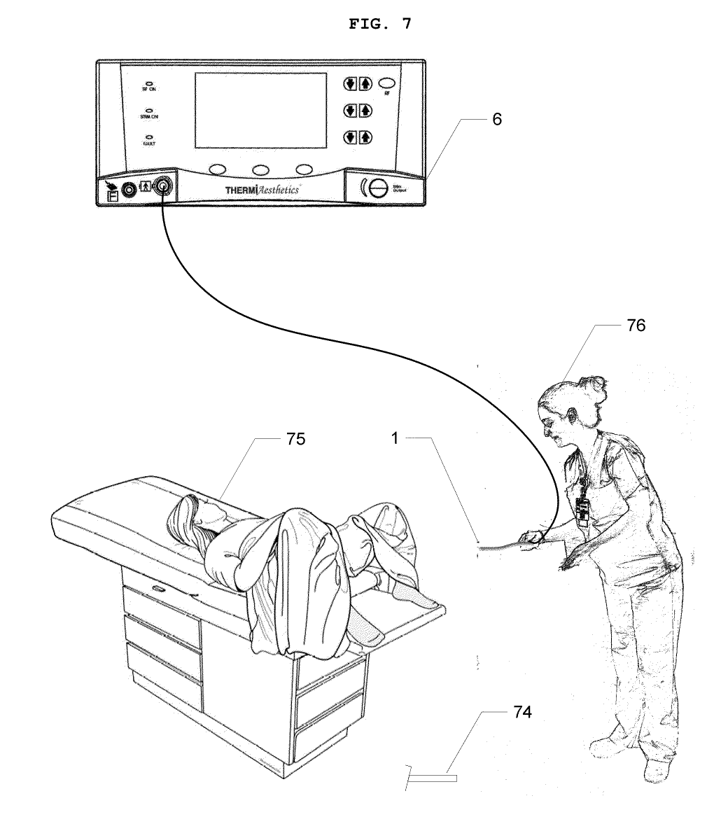

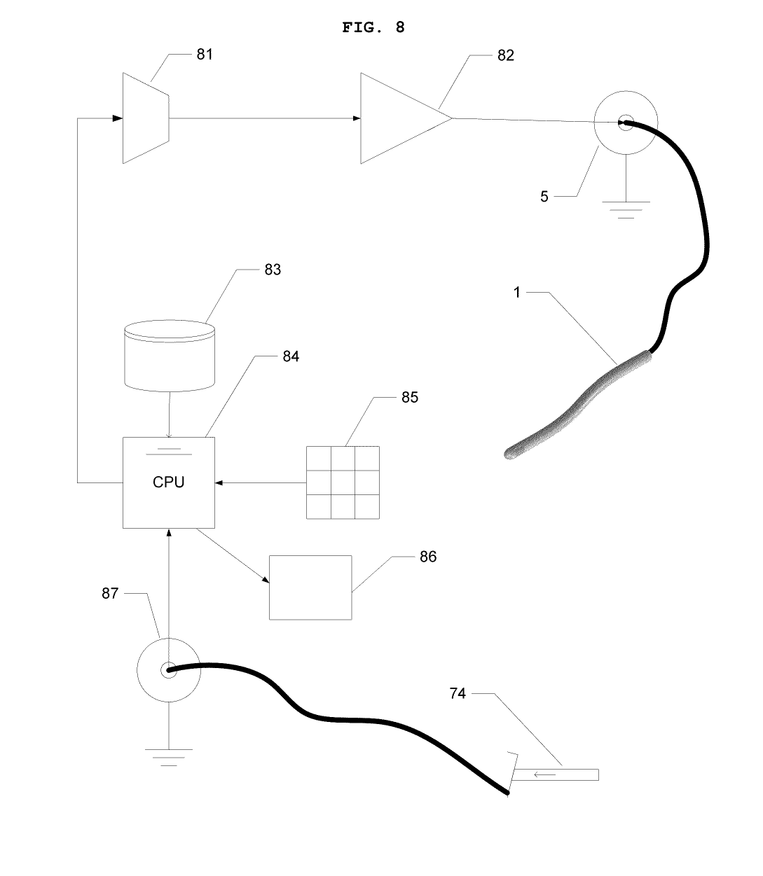

Referring to FIG. 7, a schematic of a system of treating vaginal laxity, according to an embodiment of the present invention, is now described in detail. The system as used by a user comprises an RF Source 6, a Treatment Probe 1, a Foot Pedal 74, and a Provider 76. This document will use ‘Treatment Probe’ and ‘Interior Portion’ interchangeably, as the Treatment Probe of FIG. 7 and FIG. 8 is one embodiment of the Interior Portion of FIG. 1.

As already noted, the RF Generator 6 is the RF Source 6 referenced in FIG. 1 and shown in FIG. 6. The Interior Portion 1 is the Interior Portion of FIG. 3A and FIG. 3B as well as the Interior Portion 1 of FIG. 1. The RF Generator 6 provides power and RF energy to the Interior Portion 1. The Provider 76 applies the treatment probe to the vaginal canal of the user. Multiple appointments may be made to repeat the procedure until desired results are achieved.

The RF Electrode Assembly 3 is a thin walled conductive material. This thin wall differs from other offerings in the industry which use a solid tip. The thin wall requires less time to bring the tip temperature up to skin temperature, which allows the temperature measuring means to be placed on the back of the RF surface. In the current embodiment, the thin wall is created from stainless steel, brass or copper (with a non-corrosive plating such as gold, to prevent corrosion).

The RF Electrode Assembly 3 may also includes multiple RF surfaces. One large surface may not come up to temperature ever when using low power systems like ThermiRF. Therefore an array of surfaces can be sequentially activated to threat.

Treatment may start with the labia majora, if indicated, then may proceed into the introitis and deeper vagina. In the current embodiment, a vaginal treatment area for procedure covers approximately 20+ cm² based on a vaginal circumference at a hymenal ring of approximately 12 cm.

In one embodiment, for treatment of the labia majora, a treatment tip may be applied across an entire anatomical region. The treatment tip may be applied to the labia majora bilaterally from a lowest edge of a mons pubis to a perineal body, to the labia minora and clitoral hood regions, as well as the inner labia minora tissues and vestibule, as well as the “G-Spot” where clitoral nerve fibers and branches reside.

Also treated are the periurethral tissues and saddlebag areas of the glans clitoris surrounding the urethra, which is key to treating orgasmic dysfunction) to achieve vulvar and perineal temperatures of about 40-45° C. for approximately five minutes or more of total heat time per area. The labia majora and perineal areas may take approximately 10-15 minutes to complete. This may be followed by treating a mucosal surface of the vaginal introitis starting at the hymenal ring and advancing to approximately 4-12 cm into the vaginal cavity with treatments occurring along vaginal walls, floor, and ceiling. The entire vaginal area may be treated, including the anterior wall and G-Spot areas, and treatment may consist of approximately 0.5 cm overlapping intervals, both circular and in-and-out motion, as the treatment tip moves in a clockwise and counter-clockwise direction avoiding the urethra.

This process may be repeated multiple times to achieve sustained vaginal temperature endpoints between approximately 40-45 degrees Celsius for approximately 5 minutes or longer per area depending on heat tolerance. Total vaginal treatment may be approximately 15-20 minutes.

The invention can be used to treat mild to moderate pelvic organ prolapse, which includes cystocele, rectocele and enterocoele. The RF shrinks the vaginal tissues and makes the prolapsing bulges smaller and less symptomatic. In testing, treatment with the invention has completely resolved issued regarding a fallen bladder for a patient with moderate prolapse, a result far superior to treatment using existing instruments.

One of ordinary skill in the art will appreciate that elements of this procedure may be modified while achieving a similar desired result. One of ordinary skill in the art will also appreciate that this procedure is offered as an embodiment of a method for treating vaginal laxity and not intended to limit other methods of using the systems described herein.

Referring to FIG. 8, a schematic of electrical components of the RF generator and associated system, according to an embodiment of the present invention, is now described in detail. In one embodiment, the RF generator may be an intelligent device comprising a Central Processing Unit (hereinafter referred to as “CPU”) 84, Input Keys 85, a Display 86, an RF Oscillator 81, and an RF Amplifier 82. In one embodiment of the invention, as discussed hereinabove, the RF generator may be operated by a Foot Pedal 74.

The RF Oscillator 81 may provide energy to the Amplifier 82. The Amplifier 82 may transmit energy to a Treatment Probe 1. The Treatment Probe 1 may be electronically coupled to the RF generator by a Connector 5. The Treatment Probe 1 may provide temperature feedback to the CPU 84 through the Connector 5. A remote Foot Pedal 74 may provide input to the CPU 84 through a Foot Pedal Connector 87. One of ordinary skill in the art will recognize that there are numerous methods of providing input to a CPU that may each function in the place of the Foot Pedal 74.

The CPU 84 may comprise a central processing unit, with or without integrated support features, local random access storage, and Computer Readable Media 83. The Input Keys 85 may be a keyboard or keypad comprising any number of keys, pads, buttons, switches, or other objects for inputting data. The Display 86 may be used to display information to a person operating the RF generator. The Input Keys 85 and Display 86 may be integral to the RF generator or external. The RF Oscillator and Amplifier 82 may comprise functions internal, external, or both internal and external to the RF generator. Additionally, one of ordinary skill in the art can appreciate that any electronic device may comprise a remote control feature using a plurality of communications protocols.

The RF generator may comprise a variety of Computer Readable Media 83. Computer readable media can be any available media that can be accessed by a computer and includes both volatile and nonvolatile media, removable and non-removable media. By way of example, and not limitation, computer readable media may include computer storage media and communication media. Computer storage media includes volatile and nonvolatile, removable and non-removable media implemented in any method or technology for storage of information such as computer readable instructions, data structures, program modules or other data. Computer storage media includes, but is not limited to, RAM, ROM, EEPROM, FLASH memory or other memory technology, CD-ROM, digital versatile disks (DVD) or other optical disk storage, magnetic cassettes, magnetic tape, magnetic disk storage or other magnetic storage devices, or any other medium which can be used to store the desired information and which can be accessed by a computer. Communication media typically embodies computer readable instructions, data structures, program modules or other data in a modulated data signal such as a carrier wave or other transport mechanism and includes any information delivery media. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, radio frequency, infrared and other wireless media. Combinations of any of the above should also be included within the scope of computer readable media.

Some of the illustrative aspects of the present invention may be advantageous in solving the problems herein described and other problems not discussed which are discoverable by a skilled artisan. While the above description contains much specificity, these should not be construed as limitations on the scope of any embodiment, but as exemplifications of the presented embodiments thereof. Many other ramifications and variations are possible within the teachings of the various embodiments. While the invention has been described with reference to exemplary embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best or only mode contemplated for carrying out this invention, but that the invention will include all embodiments falling within the scope of the appended claims. Also, in the drawings and the description, there have been disclosed exemplary embodiments of the invention and, although specific terms may have been employed, they are unless otherwise stated used in a generic and descriptive sense only and not for purposes of limitation, the scope of the invention therefore not being so limited. Moreover, the use of the terms first, second, etc. do not denote any order or importance, but rather the terms first, second, etc. are used to distinguish one element from another. Furthermore, the use of the terms a, an, etc. do not denote a limitation of quantity, but rather denote the presence of at least one of the referenced item.

Thus the scope of the invention should be determined by the appended claims and their legal equivalents, and not by the examples given.

A legend of the components discussed in the application and shown on the drawings is as follows:
1 Interior Portion
2 Treatment Electrode
3 RF Electrode Assembly
4 RF Connection Handle
5 Connector
6 RF Source
10 Electrode
11 Temperature Sensor
12 RF Tip
20 Input Set Temperature
21 Sensor Line
30 Control Unit
40 Switch
50 RF Energy Input
61 RF On Light
62 STIM On Light
63 Fault Light
64 Neutral Electrode Connection Port
65 Device Connection Port
66 Display Window
67 Soft Keys
68 RF On Button
69 Up/Down Buttons
70 Stim Output Knob
74 Foot Pedal
76 Provider
81 RF Oscillator
82 RF Amplifier
83 Computer Readable Media
84 CPU
85 Input Keys
86 Display
87 Foot Pedal Connector
90 Stratified Squamos Epithelium
93 Submucosa Tissue
95 Surface.

The inventors claim:

1. A treatment probe comprising:
a. a curved, rounded treatment tip designed to accommodate vaginal anatomy;
b. an electrode assembly coupled to the treatment tip, wherein the electrode coupled to the treatment tip is configured to transfer radiofrequency energy to specific vaginal structures;
c. a temperature measuring feature coupled to the electrode assembly, wherein the temperature measuring feature coupled to the electrode assembly is configured to monitor and regulate electrode and skin temperature;
d. a connector configured to connect the treatment tip to a radiofrequency generator.

2. The treatment probe according to claim 1 further comprising a protective apparatus configured to protect the treatment tip.

3. The treatment probe according to claim 1 wherein the treatment tip is connected to the radiofrequency generator by a radiofrequency handle, and wherein the radiofrequency handle is configured to be reusable.

4. The treatment probe according to claim 1 wherein the electrode assembly comprises:
a. a conductive portion;
b. a dielectric portion;
c. a radiofrequency electrode configured to capacitively couple radiofrequency energy with vaginal tissue when at least one of the conductive portion and the dielectric portion is in contact with a skin surface; and
d. a flex circuit electronically coupled to the radiofrequency electrode in which:
i. the conductive portion comprises a plurality of voids; and
ii. the dielectric portion is configured to be positioned between the conductive portion and the skin surface when the radiofrequency electrode is positioned at the skin surface.

5. The treatment probe according to claim 4 wherein the electrode assembly further comprises a back plate and a plurality of electrical contact pads, wherein the back plate is coupled to a support structure, and the plurality of electrical contact pads are coupled to the back plate.

6. The treatment probe according to claim 5 in which the support structure comprises a first engagement member and a second engagement member, and the first and second engagement members are configured to provide engagement and disengagement with a hand-piece support structure.

7. The treatment probe according to claim 1 wherein the temperature measuring feature is configured to be positioned along a back surface of the radiofrequency electrode.

8. The treatment probe according to claim 1 wherein the temperature measuring feature is configured to detect temperature along the back side of the radiofrequency electrode.

9. The treatment probe according to claim 1, further comprising a feedback mechanism configured to integrate a central processing unit to turn off the radiofrequency electrode when temperatures exceed a preset temperature.

10. The treatment probe claim 1, wherein the protective apparatus protecting the reusable treatment handle is a thin disposable cover such as flexible latex, flexible polypropylene or solid PVC or acrylic material.

11. A treatment probe comprising:
a. a curved, rounded disposable treatment tip designed to accommodate vaginal anatomy;
b. an electrode assembly coupled to the treatment tip, wherein the electrode coupled to the treatment tip is configured to transfer radiofrequency energy to specific vaginal structures;
c. a temperature measuring feature coupled to the electrode assembly, wherein the temperature measuring feature is coupled to the electrode assembly and configured to monitor and regulate electrode and skin temperature;
d. a reusable radiofrequency handle configured to connect to the treatment tip;
e. a disposable protective apparatus configured to protect the radiofrequency handle;
f. a connector configured to connect the radiofrequency handle to a radiofrequency generator source such that the treatment tip is excited by a frequency, a power level, and pattern configured to operate within a vulvovaginal cavity for the purpose of treating vulvovaginal laxity.

12. A method of treating vaginal laxity with a treatment probe, the treatment probe comprising a treatment tip designed to accommodate vaginal anatomy, an electrode assembly coupled to the treatment tip, wherein the electrode assembly is configured to transfer radiofrequency energy to specific vaginal structures, a temperature measuring feature coupled to the electrode assembly, a radiofrequency handle configured to connect to the treatment tip, a connecter configured to connect the radiofrequency handle to a radiofrequency generator, and a protective apparatus configured to protect the radiofrequency handle, the method comprising the steps of:
a. applying the treatment tip along the specific vaginal structure;
b. repeating the applying the treatment tip along the specific vaginal structure to achieve a sustained endpoint temperature within the specific vaginal structure;
c. generating power with the radiofrequency generator, wherein the power creates an electric field strength that is configured to penetrate the specific vaginal structure through a connective submucosa tissue layer to a layer of loose connective tissue.

13. The method according to claim 12 wherein the specific vaginal structure comprises one or more elements from the following list: majora and labia minora and clitoral hood, perineum, vestibule and deeper vagina.

14. The method according to claim 16 wherein the applying the treatment tip along the specific vaginal structure comprises applying to the labia majora bilaterally from a lowest edge of a mons pubis to a perineal body and laterally to a crural fold to achieve vulvar and perineal temperatures of about 40-45° C. for approximately five minutes or more of total heat time per area.

15. The method according to claim 12 wherein the applying the treatment tip along the specific vaginal structure further comprises treating a mucosal surface of the introitus starting at a hymenal ring and advancing approximately 4-12 centimeters into a vaginal cavity, wherein the vaginal cavity comprises vaginal walls, a vaginal floor, and a vaginal ceiling, and wherein the treating comprises treating the vaginal walls, the vaginal floor, and the vaginal ceiling, which includes the anterior vaginal wall, urethra, and bladder.

16. The method according to claim 15 further comprising moving the treatment probe in a steady-paced manner along the introitus and vaginal cavity to effectively provide a complete vaginal contracture without compromising a urethra.

17. The method according to claim 1 wherein the treatment probe is rounded and curved to provide smooth steady-paced movement, wherein the smooth steady-paced movement is configured to provide improved patient comfort.
